(19) United States
(12) Patent Application Publication
(10) Pub. No.: US 2010/0229250 A1
(43) Pub. Date: Sep. 9, 2010

(54) TREATMENT AND PREVENTION OF ISCHEMIC BRAIN INJURY

(75) Inventor: Sylvain Dore, Baltimore, MD (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERSITY, Baltimore, MD (US)

(21) Appl. No.: 12/677,895

(22) PCT Filed: Sep. 22, 2008
(86) PCT No.: PCT/US08/77201

§ 371 Date: May 20, 2010

Related U.S. Application Data
(60) Provisional application No. 60/994,525, filed on Sep. 20, 2007.
(60) Provisional application No. 61/067,664, filed on Feb. 29, 2008.

(51) Int. Cl. A01K 67/00

(52) U.S. Cl. 800/9

(57) ABSTRACT

The invention provides methods of identifying agents for treating and preventing ischemic brain injury.

This application claims the benefit of and incorporates by reference Ser. No. 60/994,525 filed Sep. 20, 2007 and Ser. No. 60/067,664 filed Feb. 29, 2008.

This invention was made using funds from NIH grants NS046400 and AG022971. The government therefore retains certain rights in the invention.

### FIELD OF THE INVENTION

The invention relates to methods of identifying agents for treating and preventing ischemic brain injury.

### BACKGROUND OF THE INVENTION

Cyclooxygenase (COX)-2 inhibitors are well known for their use as anti-inflammatory agents and have also been shown to reduce the damage associated with neuroinflammatory disorders. They act by inhibiting the production of prostaglandins, many of which mediate the inflammatory response. Unfortunately, COX-2 inhibitors have also been found to increase the risk of cardiovascular disease and stroke.

In most Western populations, stroke is the third leading cause of death and physical disability, after coronary heart disease and cancer (Shinohara, 2006). Animal models of transient cerebral ischemia-reperfusion (I/R) imply that brain lipids are highly prone to oxidative damage, inflammation, and apoptosis (Manabe et al., 2004). Numerous studies have consistently demonstrated that oxidative stress in stroke is caused by increased glutamate release, intracellular Ca²⁺ accumulation, and edema (Endres and Dirnagl, 2002; Xu et al., 2005). The consequent excess glutamate and hyper-activation of its receptors result in activation of phospholipid enzymes, phospholipid hydrolysis, and arachidonic acid (AA) release (Dore et al., 2003; Kawano et al., 2006; Muralikrishna Adibhatla and Hatcher, 2006).

Prostaglandins (PGs) are synthesized from arachidonic acid as a result of the consecutive actions of cyclooxygenase (COX) and PG synthesis enzymes. The effects of PGs are complex, and the brain's response is diverse because of the different types of receptors that mediate PG activity. The five primary prostanoids (PGD₂, PGE₂, PGF_{2α}, PGI₂, and TXA₂) mediate their effects mainly through their respective specific G protein-coupled receptors termed DP, EP (EP1-4), FP, IP, and TP. Receptor binding leads to either activation or inhibition of adenylyl cyclase, stimulation of phospholipase C-induced phosphoinositide turnover, mobilization of intracellular Ca2+, or stimulation of mitogen-protein kinase and protein kinase C, depending on the receptor being activated by a selective ligand (Coleman et al., 1994; Narumiya et al., 1999; Sharif et al., 2003).

PGF2α, which is synthesized from PGH₂ via PGF synthase (Suzuki-Yamamoto et al., 1999) plays an important role in initiation of parturition (Sugimoto et al., 1997), renal function (Breyer and Breyer, 2001), regulation of intraocular pressure (Ota et al., 2005), neuronal hypoxia in cultures (Li et al., 2007), control of cerebral blood flow autoregulation in newborn piglets (Chemtob et al., 1990b), contraction of arteries (Nakahata et al., 2006), and myocardial dysfunction (Takayama et al., 2005; Jovanovic et al., 2006). Several selective agonists, such as latanoprost, have been developed for the PGF2α FP receptor and are used clinically to treat ocular hypertension and glaucoma (Stjernschantz and Alm, 1996; Alexander et al., 2002; Perry et al., 2003). FP receptors have been previously demonstrated in mouse brains (Muller et al., 2000) and in brain synaptosomes of newborn pigs (Li et al., 1995). Although the critical biological functions listed above for PGF_{2α} are reported to be mediated through the FP receptor, the contribution of FP receptors in brain injury has not been investigated.

### BRIEF DESCRIPTION OF THE DRAWINGS

FIG. 1. Genetic deletion of the FP receptor does not significantly alter cerebral vasculature. Macroscopic analysis of cerebral arterial vasculature revealed no differences in the circle of Willis or major cerebral arteries between FP^{-/-} and WT mice (n=3/group). Data are shown as mean±S.E.M.

FIGS. 2A-C. FP receptor deletion does not affect physiological parameters. (FIG. 2A) Relative cerebral blood flow (CBF), (FIG. 2B) core body temperature, and (FIG. 2C) mean arterial blood pressure (MABP) were recorded at baseline, at induction of ischemia, and at 15-min intervals during ischemia and 1 h of reperfusion in WT and FP^{-/-} mice (n=5/group). Change in CBF was recorded as a percent of baseline. Data are shown as mean±S.E.M.

FIGS. 3A-C. Effect of FP receptor deletion on neurological score and infarct volume. Mice were subjected to 90-min MCAO and tested for neurological deficit at 96 h. After the testing, mice were sacrificed and brain infarction was estimated by TTC staining (FIG. 3A) Neurological deficit scores at 96 h after ischemia were significantly lower in FP^{-/-} mice than in WT mice, indicating lesser neurological dysfunction. Values are shown as mean±S.E.M. (FIG. 3B) Representative photographs show the infarcted brain slices from WT (left) and FP^{-/-} (right) mice. (FIG. 3C) Histogram shows the corrected hemispheric infarct volume of WT and FP^{-/-} mice. The infarct size (shown as mean±S.E.M) was significantly smaller in FP^{-/-} than in WT mice; *p<0.05.

FIGS. 4A-B. FP receptor knockout decreases NMDA-induced neurotoxicity. WT (n=7) and FP^{-/-} (n=7) mice were injected stereotactically in the striatum with 15 nmol NMDA and sacrificed after 48 h. Brain sections were stained with Cresyl violet and analyzed for lesions. (FIG. 4A) Representative photographs of coronal sections from the brains of WT (left panel) and FP^{-/-} (right panel) mice after intrastriatal injection with 15 nmol NMDA. The brain sections from the FP^{-/-} mouse show attenuation in lesion volume. (FIG. 4B) Histograms show that the FP^{-/-} mice were less vulnerable to the NMDA-induced neurotoxicity than were the WT mice. Values are reported as means±S.E.M.; *p<0.05, when compared with WT group.

FIGS. 5A-C. Post-treatment of mice with the FP agonist latanoprost aggravates neurological deficit and brain infarction. Mice were subjected to MCAO for 90 min and then divided into four groups: WT+vehicle (n=6), WT+10 μg/kg latanoprost (n=10), WT+100 μg/kg latanoprost (n=8), and FP^{-/-}+100 μg/kg latanoprost (n=6). Injections of vehicle or latanoprost were given at 30 min of reperfusion. Neurological deficit was determined at 96 h before the mice were sacrificed. (FIG. 5A) Histograms show the neurological scores (mean±S.E.M) of WT and FP^{-/-} mice post-treated with latanoprost. 100 μg/kg latanoprost significantly increased the neurological deficit in WT mice but not in FP^{-/-} mice. (FIG. 5B) Representative photographs of coronal sections show brain infarction in (left to right) WT mice treated with vehicle, 10 μg/kg latanoprost, 100 μg/kg latanoprost, and FP^{-/-} mice treated with 100 μg/kg latanoprost. (FIG. 5C) Latanoprost-treated WT mice had significantly larger infarct volumes than did the vehicle-treated mice, whereas no effect was observed in FP^{-/-} mice. Values are shown as mean±S.E.M; *p<0.05 compared with the WT control.

### SUMMARY OF THE INVENTION

On embodiment of the invention is a method for identifying an agent that may be useful in the treatment or prevention of ischemic brain injury. The method comprises contacting an FP receptor polypeptide and PGF2α with a test compound under conditions where the PGF2α binds to the FP receptor polypeptide; determining whether the test compound disrupts binding of the PGF2α to the FP receptor polypeptide; and determining whether the test compound prevents or decreases a symptom of ischemic brain injury in an animal model.

Another embodiment of the invention is a method for identifying an agent that may be useful in the treatment or prevention of ischemic brain injury. The method comprises contacting an FP receptor polypeptide with a test compound; determining whether the test compound binds to the FP receptor polypeptide; and determining whether the test compound prevents or decreases a symptom of ischemic brain injury in an animal model.

Another embodiment of the invention is a method for identifying an agent that may be useful in the treatment or prevention of ischemic brain injury. The method comprises contacting PGF2α with a test compound; determining whether the test compound binds to the GF2α; and determining whether the test compound prevents or decreases a symptom of ischemic brain injury in an animal model.

Yet another embodiment of the invention is method for identifying an agent that may be useful in the treatment or prevention of ischemic brain injury. The method comprises contacting an FP receptor polypeptide and PGF2α with a test compound under conditions where the PGF2α binds to the FP receptor polypeptide and induces PGF2α-mediated signaling of the FP receptor polypeptide; determining whether the test compound disrupts the PGF2α-mediated signaling; and determining whether the test compound prevents or decreases a symptom of ischemic brain injury in an animal model.

### DETAILED DESCRIPTION OF THE INVENTION

The invention provides methods for identifying agents which disrupt PGF2α-mediated signaling of the FP receptor for use in treating and preventing ischemic brain injury. The methods can be carried out in vivo or in vitro. As discussed in the Examples below, stimulating the FP receptor with an agonist (latanoprost) increases brain damage in mice due to ischemia-reperfusion injury. Conversely, mice with a genetic deletion of the FP receptor have smaller regions of injury as a result of excitotoxicity and transient ischemia than do normal (wild-type) mice. These data indicate that blocking the FP receptor either before or after an ischemic or hypoxic (or neurotoxic) event will reduce neurological-associated injury. This strategy may also be useful for reducing neurological damage associated with ischemic and hemorrhagic stroke, global ischemia, vascular dementia, Alzheimer's disease, and other acute or chronic neurological disorders. See Connolly et al., Cogn Behav Neurol. 2008 June; 21(2):83-6; Kim et al., Brain Res Bull. 2004 Jul. 30; 64(1):47-51; Yao et al., Neurology. 2003 Aug. 26; 61(4):475-8; Asaeda et al., Neurosci Lett. 2005 Jan. 20; 373(3):222-5; Ogawa et al., Acta Neuropathol. 1988; 76(5):496-501; and Ogawa et al., Acta Neuropathol. 1987; 75(1):62-8.

Agents can disrupts PGF_{2α}-mediated signaling of the FP receptor in a variety of ways. In some embodiments, the agent prevents or reduces binding of PGF_{2α} to the FP receptor. Alternatively or additionally, the agent may affect the interaction between PGF_{2α} and the FP receptor, or the interaction between the FP receptor and the associated Gαq protein, thus inhibiting or disrupting the PGF_{2α}-FP mediated signal transduction pathway.

In other embodiments, the agent is an antagonist of the FP receptor. FP receptor antagonists are typically molecules which bind to the FP receptor, compete with the binding of the natural ligand PGF_{2α}, and inhibit or disrupt the PGF_{2α}-FP mediated signal transduction pathway. Such antagonists preferably are selective for the FP receptor and preferably have an equal or higher binding affinity to the FP receptor than does PGF_{2α}. Although antagonists with a higher affinity for the receptor than the natural ligand are preferred, antagonists with a lower affinity may also be useful. Preferably, antagonists bind reversibly to the FP receptor.

In some embodiments, the agent occupies the PGF_{2α} binding site on the prostaglandin receptor, such that the natural ligand (PGF_{2α}) is prevented from binding in a mode that would result in its normal mode of signaling via Gq/Gq_{II} through inositylphosphate and subsequent mobilisation of intracellular calcium.

Alternatively, the agent may bind to the FP receptor without preventing PGF_{2α} binding to the receptor, but which disrupts the interaction between PGF_{2α} and the FP receptor, thus inhibiting or disrupting PGF_{2α}-FP mediated signal transduction pathway.

In other embodiments, an FP receptor antagonist binds to the FP receptor and disrupts the interaction between the FP receptor and the associated Gα_{q} protein, thus inhibiting or disrupting FP mediated signal transduction pathway.

In other embodiments, the agent is an antagonist of PGF₂α. PGF_{2α} antagonists are typically molecules which bind to PGF_{2α} and prevent or reduce PGF_{2α} binding to its receptor, which inhibits or disrupts the PGF_{2α}-FP mediated signal transduction pathway. This is the “soluble receptor” approach in which typically either a part of the receptor or an antibody binds to PGF_{2α}. Alternatively, the PGF_{2α} antagonist may be a molecule which binds to PGF_{2α} without preventing or reducing the binding of PGF_{2α} to the FP receptor, but which disrupts the interaction between PGF_{2α} and the FP receptor such that the PGF_{2α}-FP mediated signal transduction pathway is inhibited or disrupted. This could be a molecule which binds in a covalent fashion to PGF_{2α} and has no effect on binding potency but effects the G-protein/IP/Ca²⁺ mechanisms.

### Test Compounds

According to the present invention, test compounds are tested for the ability to disrupt PGF_{2α}-mediated signalling of the FP receptor. Test compounds for use in methods of the invention can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art.

Test compounds can be obtained using any of the numerous combinatorial library methods known in the art, including but not limited to, biological libraries, spatially addressable parallel solid phase or solution phase libraries, synthetic library methods requiring deconvolution, the “one bead one compound” library method, and synthetic library methods using affinity chromatography selection. Methods for the synthesis of molecular libraries are well known in the art (see, for example, DeWitt et al., Proc. Natl. Acad. Sci. U.S.A. 90, 6909, 1993; Erb et al., Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-21, 1992), or on beads (Lam, Nature 354, 82-84, 1991), chips (Fodor, Nature 364, 555-56, 1993), bacteria or spores (Ladner, U.S. Pat. No. 5,223,409), plasmids (Cull et al., Proc. Natl. Acad. Sci. U.S.A. 89, 1865-869, 1992), or phage (Scott & Smith, Science 249, 386-90, 1990; Devlin, Science 249, 404-06, 1990); Cwirla et al., Proc. Natl. Acad. Sci. 97, 6378-82, 1990; Felici, J. Mol. Biol. 222, 301-10, 1991; and Ladner, U.S. Pat. No. 5,223,409).

### FP Receptor Polypeptides

FP receptor polypeptides for use in screening methods of the invention can be produced recombinantly, purified from natural sources, or synthesized chemically. The amino acid sequences of FP receptors from various species such as human (GenBank Accession No. NM_{—}000959), mouse (GenBank Accession No. P43117), rat (GenBank Accession No. NP_{—}037247), cat (GenBank Accession No. AAL36977), sheep (GenBank Accession No. Q28905), cow (GenBank Accession No. BAA20871), and monkey (GenBank Accession No. AAB36298) are known in the art. FP receptor protein can be produced using routine expression methods, for example in E. coli or CHO cells comprising an expression vector encoding the FP receptor protein. Using well-known recombinant DNA methods, a recombinant DNA containing the cDNA for FP receptors can be constructed and transformed into a host cell, which can then cultured to produce the enzyme. See US 2006/0105346. “FP receptor polypeptide” as used herein encompasses both full-length FP receptors and soluble forms of the receptors which retain the ability to bind PGF_{2α}.

FP receptors can be purified from the culture by conventional means. FP receptors also can be purified from natural sources (e.g., eye, small intestine, corpus luteum, placenta, ovary, brain, myometrium, lung, kidney, stomach, muscle, uterus, trachea, corpus luteum) using methods well known in the art. Such methods include, but are not limited to, size exclusion chromatography, ammonium sulfate fractionation, ion exchange chromatography, affinity chromatography, and preparative gel electrophoresis.

An FP receptor can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995), which is incorporated herein by reference in its entirety. Protein synthesis can be performed using manual techniques or by automation. Automated synthesis can be achieved, for example, using Applied Biosystems 431A Peptide Synthesizer (Perkin Elmer). Optionally, fragments of an FP receptor can be separately synthesized and combined using chemical methods to produce a full-length molecule. See WO 01/98340.

PGF_{2α} can be purchased from various companies such as Cayman Chemical, Ann Harbor Michigan (PGF2α; Dinoprost, CAS 551-11-1); alternatively, relatively selective agonists such as Fluprostenol and Latanoprost can be used.

### Binding Assays

Binding of a test compound to PGF_{2α} or an FP receptor polypeptide can be detected using a variety of methods. Displacement experiments, for example, can be carried out using cells which express an FP receptor polypeptide. The cells are incubated in a binding buffer with labeled PGF_{2α} in the presence or absence of increasing concentrations of a test compound. To validate and calibrate the assay, control competition reactions using increasing concentrations of unlabeled PGF_{2α} can be performed. After incubation, cells are washed extensively, and bound, labeled PGF_{2α} is measured as appropriate for the given label (e.g., scintillation counting, fluorescence, etc.). A decrease of at least 10% in the amount of labeled PGF_{2α}, bound in the presence of a test compound indicates displacement of PGF_{2α} binding by the test compound. Test compounds are considered to bind specifically in this or other assays if they displace 50% of labeled PGF_{2α} at a concentration of 1 mM or less.

Alternatively, binding or displacement of binding can be monitored by surface plasmon resonance (SPR). Surface plasmon resonance assays can be used as a quantitative method to measure binding between two molecules by the change in mass near an immobilized sensor caused by the binding or loss of binding of PGF_{2α} from the aqueous phase to an FP receptor polypeptide, the binding of a candidate molecule from the aqueous phase to the GPCR. This change in mass is measured as resonance units versus time after injection or removal of the PGF₂a or the test compound and can be measured using a Biacore Biosensor (Biacore AB).

An FP receptor polypeptide can be immobilized on a sensor chip (for example, research grade CM5 chip; Biacore AB) in a thin film lipid membrane (Salamon et al., Biophys J. 71, 283-94, 1996; Salamon et al., Biophys. J. 80, 1557-67, 2001; Salamon et al., 1999, Trends Biochem. Sci. 24: 213-19, 1999) or immobilized in a lipid layer on the chip (Sarrio et al., 2000, Mol. Cell. Biol. 20, 5164-74, 2000). Conditions for test compound binding to FP receptor polypeptides in an SPR assay can be fine-tuned by one of skill in the art using the conditions reported by Sarrio et al. as a starting point.

SPR can be used to assay for competitors of PGF_{2α} binding in at least two ways. First, PGF_{2α} can be pre-bound to an immobilized FP receptor polypeptide, followed by injection of a test compound at a concentration ranging from 0.1 nM to 1 μM. Displacement of the bound PGE₂ can be quantitated, permitting detection of modulator binding. Alternatively, the membrane-bound FP receptor polypeptide can be pre-incubated with a test compound and challenged with PGF_{2α}. A difference in PGF_{2α} binding to the FP receptor polypeptide exposed to test compound relative to that on a chip not pre-exposed to the test compound will demonstrate binding or displacement of PGF_{2α} in the presence of test compound. In either assay, a decrease of 10% or more in the amount of PGF_{2α} bound in the presence of a test compound relative to the amount of PGF_{2α} bound in the absence of the test compound indicates that the test compound inhibits the binding of PGF_{2α} to the FP receptor polypeptide.

In binding assays, either the test compound, PGF_{2α} or an FP receptor polypeptide can comprise a detectable label, such as a fluorescent, radioisotopic, chemiluminescent, or enzymatic label. Examples of detectable labels include horseradish peroxidase, alkaline phosphatase, and luciferase. Detection of a test compound that is bound to PGF_{2α} or an FP receptor polypeptide can then be accomplished, for example, by direct counting of radioemmission, by scintillation counting, or by determining conversion of an appropriate substrate to a detectable product.

In yet another aspect of the invention, PGF_{2α} or an FP receptor polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with PGF_{2α} or an FP receptor polypeptide.

Another method of detecting inhibition of binding of PGF_{2α} to an FP receptor polypeptide uses resonance energy transfer methods, such as FRET (fluorescence resonance energy transfer) or LRET (luminescence resonance energy transfer). Resonance energy transfer is a quantum mechanical phenomenon that occurs between a donor (D) and an acceptor (A) in close proximity to each other (usually <100 A of separation) if the emission spectrum of D overlaps with the excitation spectrum of A. The molecules to be tested, e.g. PGF_{2α} and an FP receptor polypeptide, can be labeled with a complementary pair of donor and acceptor moieties. While bound closely together by the PGF_{2α}-FP receptor polypeptide interaction, the energy emitted upon excitation of the donor moiety will have a different wavelength than that emitted in response to that excitation wavelength when the PGF_{2α} and FP receptor polypeptide are not bound. This permits quantitation of bound versus unbound molecules by measurement of emission intensity at each wavelength. Properties which can be detected as resonance energy transfer measurements include a molar extinction coefficient at an excitation wavelength, a quantum efficiency, an excitation spectrum, an emission spectrum, an excitation wavelength maximum, an emission wavelength maximum, a ratio of excitation amplitudes at two wavelengths, a ratio of emission amplitudes at two wavelengths, an excited state lifetime, anisotropy, a polarization of emitted light, resonance energy transfer, and a quenching of emission at a wavelength.

Functional Assays

Binding of a prostaglandin agonist such as PGF_{2α} to an FP receptor polypeptide can activate numerous intracellular effector systems including, without limitation, the trimeric G-proteins Gα_{q} and Gα₁₁ (Carrasco et al., J. Repr. Fertil. 111:309-17, 1997), the small G-protein Rho (Pierce et al., J. Biol. Chem. 274:35944-49, 1999), phospholipase C (Gusovsky, Mol. Pharmacol. 40:633-38, 1991); Boiti et al., J. Endocrinol. 164:179-86, 2000), inositol triphosphate/free intracellular calcium (Davis et al., Proc. Natl. Acad. Sci. USA 84:3728-32, 1987; Wiltbank et al., Biol. Reprod. 41:771-778, 1989), phospholipase D (Liu et al., Prostaglandins 51:233-48, 1996), and mitogen-activated protein kinases (Chen et al., Endocrinology 139:3876-85, 1998; Niswender et al., Physiol. Rev. 80:1-29, 2000). Assays for any of these activities can be used to determine whether a compound disrupts PGF2α-mediated signaling of an FP receptor polypeptide.

Assays for these activities are well known in the art and include, for example, PGF2α stimulation of total InsP production as described in US 20070004620. Intracellular calcium can be monitored using various using electrophysiology and an aequorin luminescence assay. Also the calcium downstream cascade can also be investigated. Phosphoinositide (PI) levels and phospholipase C activity can be measured. Finally the primary effector of the FP stimulation would lead to regulation of Gq/G¹¹ family, such that the G-protein regulated pathways can also be investigated. See also Brown et al., Methods Enzymol. 2007; 434:49-87 (phospholipase D); Caelles & Morales, Methods in Molecular Biology, Vol. 282; pp 145-156. Ed: H. J. M. Brady; Humana Press 2004 (MAPK activity); Pellegrin & Mellor, Curr Protoc Cell Biol. 2008 March; Chapter 14:Unit 14.8 (Rho activity); WO/2003/038039; and Eglen, Frontiers in Drug Design & Discovery; 2005; 1:97-111.

### High Throughput Screening

Screening methods of the invention can be used in high through-put screening formats. Using high throughput screening, many discrete compounds can be tested in parallel so that large numbers of test compounds can be quickly screened. The most widely established techniques utilize 96-well microtiter plates, however 384- or 1536-plates also can be used. As is known in the art, a variety of instruments, materials, pipettors, robotics, plate washers, and plate readers are commercially available.

### Animal Models

After a test compound is identified as able to block or antagonize an FP receptor, the test compound is administered to an animal model to determine whether it can reduce or prevent a symptom of brain ischemia. Animal models of brain ischemia are well known in the art. A useful model is described in Example 1. Symptoms that can be assessed include infarct volume, limb weakness, torso turning to the ipsilateral side of the lesion, circling to the affected side, ability to bear weight on the affected side, degree of locomotor activity or barrel rolling, mean arterial blood pressure, and blood chemistry (e.g., pH, PaCO₂, PaO₂). See Example 1. A test compound preferably reduces one or more of these symptoms by 10, 20, 30, 40, 50, 60, 70, 80, 90, or 100%.

Animal models for neurological disorders, such as ischemic and hemorrhagic stroke, global ischemia, vascular dementia, Alzheimer', Parkinson, and other acute or chronic neurological disorders are well known in the art

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLES

This study was specifically designed to investigate the effect of the FP receptor on neurobehavioral outcomes and infarct volume in a transient ischemia model of stroke in mice. Another set of experiments was conducted to further determine the role of FP in NMDA-induced excitotoxicity. We found that the resulting infarct volume after I/R injury was significantly smaller in mice lacking the FP receptor than in WT mice, while other physiological parameters monitored were unaffected. Unilateral induction of excitotoxicity also caused a significantly smaller lesion volume in FP^{-/-} than in WT mice. Finally, post-treatment of WT mice with the FP agonist latanoprost 30 min after the start of reperfusion significantly increased the neurological deficit and brain injury size as compared with those of the vehicle-treated group. The results suggest that FP receptors significantly contribute to exacerbation of excitotoxic and transient ischemic brain damage.

### Example 1

### Methods

Animals. This experiments described below were carried out in accordance with the National Institutes of Health guidelines for the use of experimental animals. Protocols were approved by the Johns Hankins Institutional Animal Care and Use Committee. FP^{-/-} C57BL/6 breeding mice were first obtained from Dr. Narumiya (Sugimoto et al., 1997) and then maintained and genotyped at the animal facility at Johns Hopkins University. Adult male FP^{-/-} and WT mice (20-25 g; 8-10 weeks old) were used in this study. All measures were taken to minimize pain and discomfort to the mice.

Evaluation of cerebral vessel diameter. Because anatomical differences in the cerebral vasculature might affect the stroke outcome, we examined the anatomy of the large vessels in WT and FP^{-/-} mice. Mice (n=3) from each genotype were deeply anesthetized with pentobarbital sodium (65 mg/kg i.p.) and transcardially perfused with 5 ml of ice-cold saline followed by 1 ml of black latex paint. The mice were decapitated and their brains carefully removed and immersed in 10% formalin for 24 h before examination. The vessel diameters were evaluated with Metavue software (Meta Imaging Series Software, Downingtown, Pa.).

Transient ischemia protocol. WT (n=11) and FP^{-/-} (n=12) mice were subjected to MCAO for 90 min followed by 96 h of reperfusion (Ahmad et al., 2006a). Mice were placed under halothane anesthesia (3.0% for induction, 1.0% for maintenance) and ventilated with oxygen-enriched air via a nose cone. Body temperatures were maintained at 37.0±0.5° C. by a heating pad. The mice were subjected to the intraluminal filament technique to produce the MCAO model of transient focal cerebral ischemia as we have described previously (Ahmad et al., 2006a). Relative cerebral blood flow (CBF) was measured by laser-Doppler flowmetry (Moor instruments, Devon, England) with a flexible probe affixed to the skull over the parietal cortex supplied by the MCA (2 mm posterior and 6 mm lateral to bregma). The induction of MCAO was achieved when the relative CBF decreased more than 80% from baseline; mice for which the CBF did not decrease below that level were excluded from additional experiments. During occlusion, mice were kept in a humidity-controlled, 32° C. chamber to help maintain a body core temperature of 37° C. After 90 min of occlusion, mice were briefly re-anesthetized, the midline was reopened, and the filament was removed to establish reperfusion. After the incision was sutured, mice were again placed in the humidity- and temperature-controlled chamber for another 6 h and finally returned to their respective cages for survival up to 96 h.

Measurement of physiological parameters. In a separate cohort of WT (n=5) and FP^{-/-} (n=5) mice, the femoral artery was cannulated for measurement of physiological parameters (pH, PaCO₂, and PaO₂) and mean arterial blood pressure (MABP). Physiological parameters were monitored at baseline, 1 h of ischemia, and 1 h after reperfusion, whereas CBF, MABP, and rectal temperature were monitored every 15 min before and during ischemia and for 1 h of reperfusion.

Assessment of neurological deficit score. Neurological function was measured in each mouse at 96 h after reperfusion according to the following graded scoring system, as described previously (Li et al., 2004): 0=no deficit; 1=forelimb weakness and torso turning to the ipsilateral side when held by tail; 2=circling to affected side; 3=unable to bear weight on affected side; and 4=no spontaneous locomotor activity or barrel rolling.

Post-treatment of mice with the FP agonist latanoprost. We used the selective FP receptor agonist latanoprost (13,14-dihydro-17-phenyl-18,19,20-trinor-PGF_{2α} isopropyl ester; Cayman Chemicals, Ann Arbor, Mich.), which is also used clinically (Abdel-Halim et al., 1977; Perry et al., 2003), to determine the role of FP receptor activation in ischemic brain damage. Latanoprost was supplied as a 10 mg/ml solution in methyl acetate. The methyl acetate was evaporated under nitrogen, and the residual latanoprost was immediately dissolved in 100 μl of DMSO and diluted with 0.9% saline to obtain a stock solution of 10 mg/ml. Subsequent doses were freshly made such that each mouse received a final concentration of 0.5% DMSO. WT mice were post-treated orally with 10 μg/kg (n=10) or 100 μg/kg (n=8) latanoprost 30 min after reperfusion. A vehicle-control group (n=6) was given 0.5% DMSO (diluted with 0.9% saline). At 96 h after MCAO, neurological deficit scores and brain injury were measured. To further determine the specificity of latanoprost toward stroke outcome, we repeated the experiment in FP−/− mice using 100 μg/kg latanoprost (n=6).

Assessment of brain infarction. After 96 h of reperfusion, mice were deeply anesthetized, and their brains were collected and sliced coronally into 2-mm-thick sections. Infarct volume was assessed by 2,3,5-triphenyltetrazolium chloride (TTC) staining by an observer blinded to the treatments, as described previously (Ahmad et al., 2006a). Infarct volume was corrected for swelling by comparing the volumes in the ipsilateral and contralateral hemispheres. The corrected volume of the infarcted hemisphere was calculated as: volume of contralateral hemisphere−(volume of ipsilateral hemisphere−volume of infarct).

Unilateral NMDA-induced acute excitotoxicity. To investigate the effect of FP on NMDA toxicity, WT and FP^{-/-} mice (n=7/group) were injected in the right striatum with 15 nmol NMDA (in 0.3 μl) or vehicle as described before (Ahmad et al., 2006a). After injection, the hole was blocked and the skin sutured, and the animals were transferred to a humidity- and temperature-controlled chamber. After full recovery from anesthesia, the mice were transferred to their home cages and were allowed to survive for 48 h. Throughout the stereotactic procedure, the rectal temperature of the mice was monitored and maintained at 37.0±0.5° C.

Quantification of the excitotoxic lesion volume. Forty-eight hours after NMDA injection, weight and rectal temperature were recorded, and the mice were deeply anesthetized with pentobarbital sodium (65 mg/kg). The mice then were perfused transcardially with cold PBS followed by 4% paraformaldehyde (pH 7.2) in PBS. Brains were removed quickly and post-fixed overnight before being equilibrated in sucrose (30%) and frozen in 2-methyl butane (pre-cooled over dry ice). Sequential brain sections (25 μm) were cut on a cryostat, and every fourth section was stained with Cresyl violet to estimate lesion volume, as described previously (Ahmad et al., 2006b). Briefly, every stained section was imaged with computerized SigmaScan Pro 5.0 (Systat, Inc., Point Richmond, Calif.) software. The entire lesion (recognized as a lightly stained area with extensive cell loss in the ipsilateral striatum) was encircled, enabling the software to calculate the lesion area in pixels. The area in pixels of every stained section was summed and divided by the total number of sections to obtain the mean lesion area in pixels. A piece of graph paper was also photographed, a square area was measured in pixels to convert the lesion area from pixels to mm2. Then the lesion area in mm2 was multiplied by the section thickness to determine the lesion volume in mm3. In different groups, the number of sections with lesions ranged from 50 to 80.

Statistical analysis. The brain sections were imaged and analyzed with SigmaScan Pro 5.0 software (Systat, Inc., Point Richmond, Calif.); SigmaStat 3.11 was used for statistical analysis. We used two-way ANOVA followed by Bonferroni multiple comparison test to determine the difference in the cerebral artery diameter between the WT and FP^{-/-} mice and to determine the difference in the physiological parameters between two groups at a given time point. One-way ANOVA followed by Bonferroni multiple comparison test was used to determine the difference in brain damage and neurological deficit scores after MCAO in WT and FP^{-/-} mice, between latanoprost-treated groups, and after NMDA injection in WT and FP^{-/-} mice. All values are expressed as mean±standard error of the mean (S.E.M.). Values of p<0.05 were considered to be significant.

### Example 2

### Genetic Deletion of FP Receptors does not Affect the Cerebrovascular Anatomy or Vital Physiological Parameters in Mice

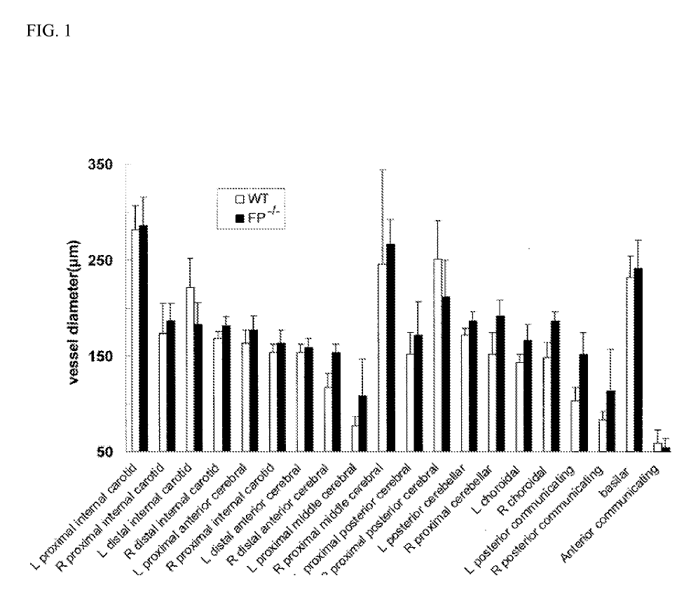

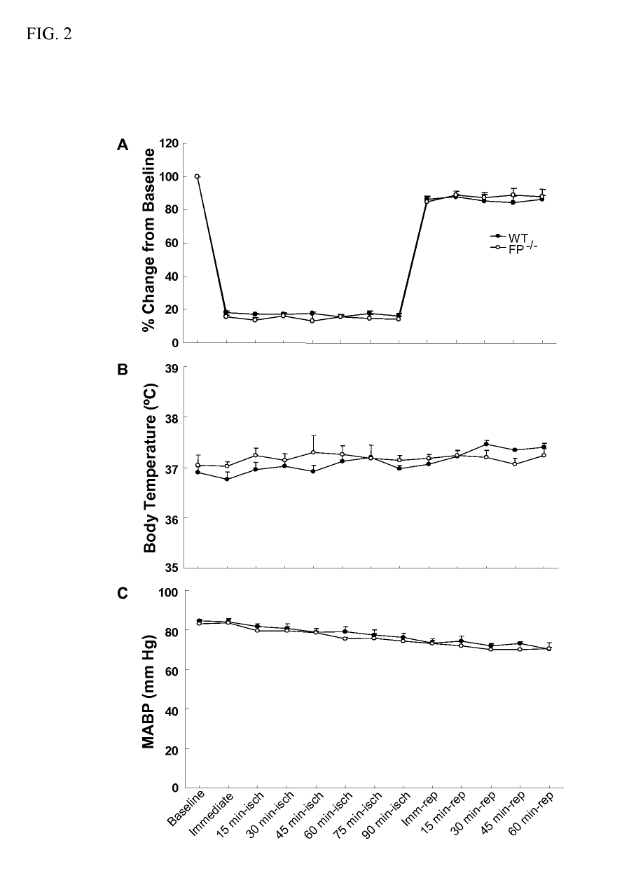

The analysis of the vascular macrostructure of the brains from WT and FP^{-/-} mice did not reveal detectable differences in the cerebral anatomy between the two genotypes (FIG. 1), suggesting that no gross blood vessel anatomical differences are apparent under these experimental conditions. This result allowed us to confidently pursue the I/R experimental paradigm. Additionally, we detected no significant differences in blood gases (_{PaO2}, PaCO₂) or pH between WT and FP^{-/-} mice during MCAO or after reperfusion (Table I). The relative CBF decreased to ≧80% from baseline in both groups after MCAO and returned to near baseline after reperfusion was achieved. No substantial differences in CBF, body temperature, or MABP were observed between the two groups before, during, or after MCAO (FIG. 2).

TABLE I
Effect of MCAO on physiological parameters in WT and FP^{-/-} mice

| | WT Mice | | | FP^{-/-} Mice | | |
|---|---|---|---|---|---|---|
| | | 1 h | 1 h | | 1 h | 1 h |
| Parameter | Baseline | MCAO | Reperfusion | Baseline | MCAO | Reperfusion |
| pH | 7.34 ± 0.02 | 7.33 ± 0.01 | 7.34 ± 0.01 | 7.34 ± 0.01 | 7.33 ± 0.02 | 7.32 ± 0.04 |
| PaCO₂ | 38.4 ± 1.1 | 40.4 ± 1.2 | 38.9 ± 1.1 | 39.0 ± 1.1 | 40.3 ± 1.0 | 40.8 ± 1.8 |
| PaO₂ | 104 ± 3 | 130 ± 3 | 106 ± 2 | 114 ± 4 | 120 ± 3 | 113 ± 8 |

### Example 3

### Reduced Neurological Deficits and Brain Infarction in FP^{-/-} Mice after Transient Ischemia

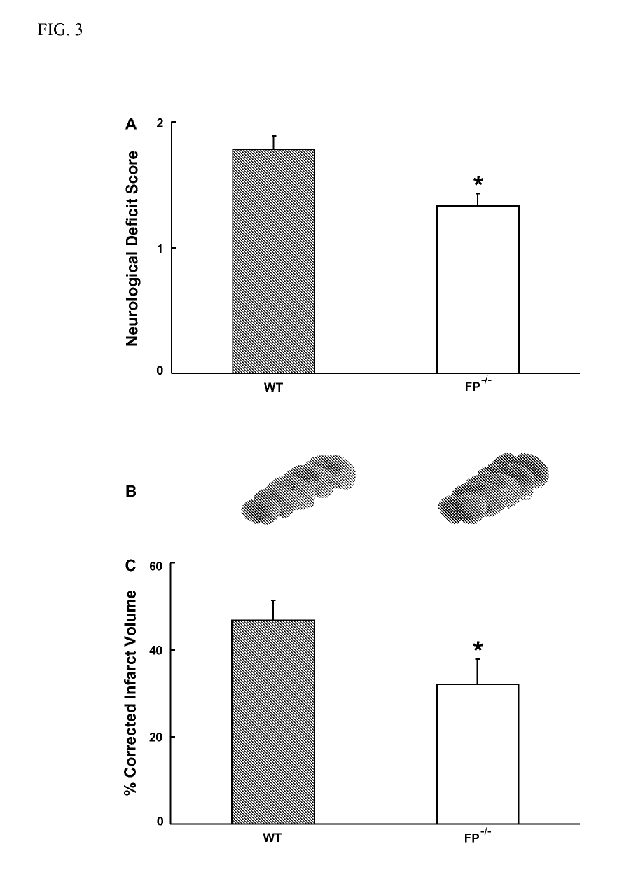

At 96 h after the occlusion, the neurological deficit scores of the FP^{-/-} mice were significantly (p<0.05) lower than those of the WT mice (FIG. 3A). In addition, analysis of the brain slices stained with TTC showed that FP^{-/-} mice had significant attenuation (p<0.05) of brain infarction volume as compared with similarly treated WT mice (FIGS. 3B, C).

### Example 4

### NMDA-Induced Neurotoxicity is Reduced in FP^{-/-} Mice

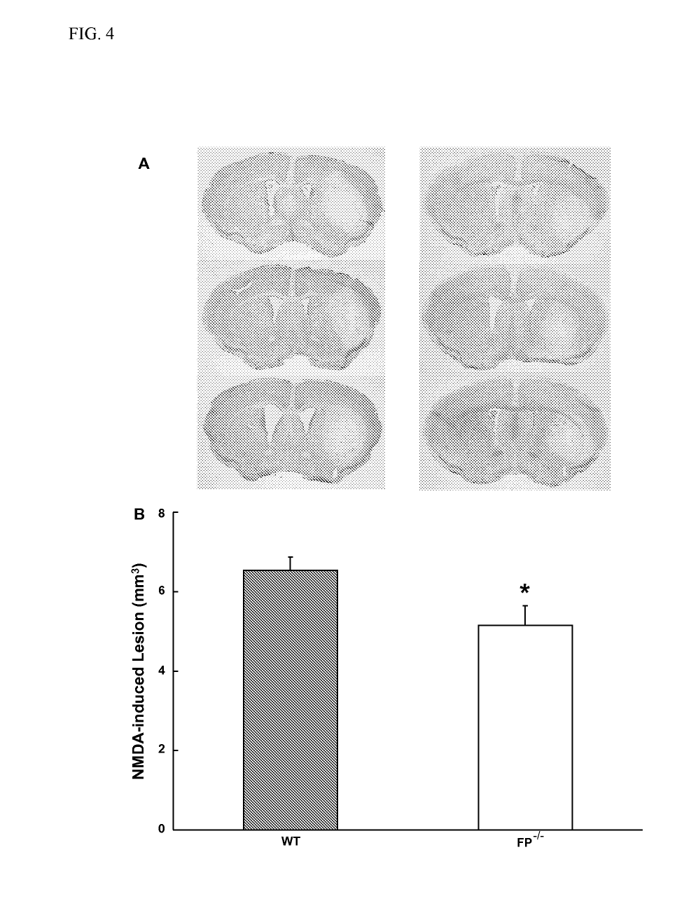

To further investigate the contribution of FP receptor in the pathology of stroke, we investigated whether FP^{-/-} mice would be protected against acute excitotoxicity induced by NMDA. Cresyl violet-stained brain sections revealed that the NMDA-induced lesion volume was significantly smaller (p<0.05) in the FP^{-/-} mice than in the WT mice (FIG. 4).

### Example 5

Activation of FP Receptor by Agonist Latanoprost Augments Stroke Outcome in WT but not in FP^{-/-} Mice

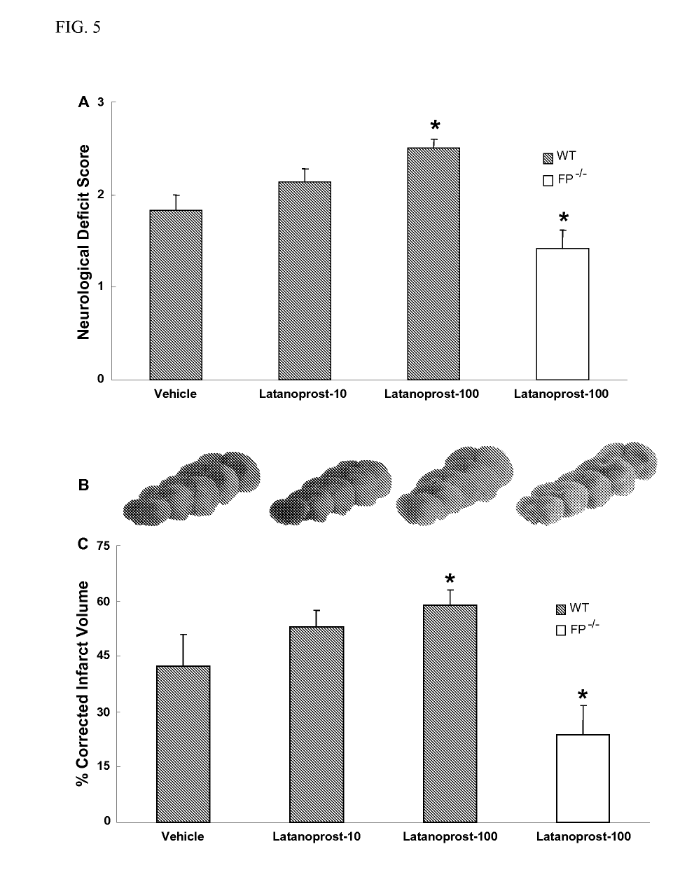

To take into consideration the potential compensatory mechanisms in knockout animals and further confirm the role of the FP receptor in stroke, we investigated the effect of post-treatment with the FP receptor agonist latanoprost in WT and FP^{-/-} mice subjected to MCAO. The WT cohort treated with 10 μg/kg latanoprost showed a trend toward increased neurological deficit (2.2±0.2), although the effect was not statistically significant when compared with the vehicle-treated mice (1.8±0.2). However, the neurological deficit increased significantly (p<0.05) in the group that was post-treated with 100 μg/kg latanoprost. Similarly, a trend toward increased brain damage was seen in mice that were post-treated with 10 μg/kg latanoprost, which became significant (p<0.05) in the group that was given 100 μg/kg latanoprost (FIG. 5). Interestingly, the deleterious effect of latanoprost was not observed in FP^{-/-} mice, which had significantly lower neurological deficit scores (1.4±0.2; p<0.05) and smaller infarct volumes than latanoprost-treated WT mice.

### REFERENCES

Abramovitz et al. (1994) Cloning and expression of a cDNA for the human prostanoid FP receptor. J Biol Chem 269:2632-2636.
Ahmad et al. (2005) Prostaglandin EP4 receptor agonist protects against acute neurotoxicity. Brain Res 1066:71-77.
Ahmad et al. (2006a) Prostaglandin EP1 receptorcontributes to excitotoxicity and focal ischemic brain damage. Toxicol Sci 89:265270.
Ahmad et al. (2006b) 1-HydroxyPGE, reduces infarction volume in mouse transient cerebral ischemia. Eur J Neurosci 23:35-42.
Ahmad et al., Stimulation of prostaglandin EP2 receptors prevents NMDA-induced excitotoxicity. J. Neurotrauma 23, 18951903.
Ahmad et al. 2006c. 1-HydroxyPGE, reduces infarction volume in mouse transient cerebral ischemia. Eur. J. Neurosci. 23, 35-42.
Alexander et al., 2002. Prostaglandin analog treatment of glaucoma and ocular hypertension. Ann Pharmacother 36, 504-511.
AI-Matubsi et al. (2001) Expression and localization of the contractile prostaglandin F receptor in pregnant rat myometrium in late gestation, labor, and postpartum. Biol Reprod 65:1029-1037.
Ayata et al., 1997. Mechanisms of reduced striatal NMDA excitotoxicity in type I nitric oxide synthase knock-out mice. J. Neurosci. 17, 6908-6917.
Bano & Nicotera (2007) Ca2+ signals and neuronal death in brain ischemia. Stroke 38:674-676.
Brault et al. (2003) Selective neuromicrovascular endothelial cell death by 8-Iso-prostaglandin F2alpha: possible role in ischemic brain injury. Stroke 34:776-782.
Breyer & Breyer, 2001. G protein-coupled prostanoid receptors and the kidney. Annu Rev. Physiol. 63, 579-605.
Chemtob et al. (1990) Changes in cerebrovascular prostaglandins and thromboxane as a function of systemic blood pressure. Cerebral blood flow autoregulation of the newborn. Circ Res 67:674682.
Chemtob, S., Beharry, K., Rex, J., Varma, D. R., Aranda, J. V., 1990b. Prostanoids determine the range of cerebral blood flow autoregulation of newborn piglets. Stroke 21, 777-784.
Cheng et al. (2006) Antagonism of the prostaglandin D2 receptor 1 suppresses nicotinic acid-induced vasodilation in mice and humans. Proc Natl Acad Sci USA 103:6682-6687.
Choi, D. W., 1992. Excitotoxic cell death. J Neurobiol 23, 1261-1276.
Coleman et al. (1994) International Union of Pharmacology classification of prostanoid receptors: properties, distribution, and structure of the receptors and their subtypes. Pharmacol Rev 46:205-229.
Corbett et al. (1997) Hypothermic neuroprotection. A global ischemia study using 18- to 20-month-old gerbils Stroke 28:2238-2242; discussion 2243.
Cuppoletti et al. (2007) Cellular and molecular effects of unoprostone as a BK channel activator. Biochim Biophys Acta 1768:1083-1092.
Dore et al. (2003) Neuronal overexpression of cyclooxygenase-2 increases cerebral infarction. Ann Neurol 54:155-162.
Dietrich & Bramlett, 2007. Hyperthermia and central nervous system injury. Prog Brain Res 162, 201-217.
Dore et al., 2003. Neuronal overexpression of cyclooxygenase-2 increases cerebral infarction. Ann. Neurol. 54, 155-162.
Echeverria et al. (2005) Stimulation of PGE2 receptors EP2 and EP4 protects cultured neurons against oxidative stress and cell death following Ramyloid exposure. Eur J Neurosci 22:2199-2206.
Ellis et al. (1979) Vasodilation of cat cerebral arterioles by prostaglandins D2, E2, G2, and 12. Am J Physiol 237:H381-385.
Endres & Dirnagl (2002) Ischemia and stroke. Adv Exp Med Biol 513:455-473. Georgiadis D, Schwab S (2005) Hypothermia in Acute Stroke. Curr Treat Options Neurol 7:119-127.
Hackam & Spence (2007) Combining multiple approaches for the secondary prevention of vascular events after stroke: a quantitative modeling study. Stroke 38:1881-1885.
Hata & Breyer (2004) Pharmacology and signaling of prostaglandin receptors: multiple roles in inflammation and immune modulation. Pharmacol Ther 103:147166.
Heaslip & Sickels (1989) Evidence that prostaglandins can contract the rat aorta via a novel protein kinase C-dependent mechanism. J Pharmacol Exp Ther 250:44-51.
Hebert et al. CA, Qi Z, Breyer R M, Natarajan C, Hata A N, Zhang Y, Guan Y, Breyer M D (2005) Characterization of a rabbit kidney prostaglandin F(2{alpha}) receptor exhibiting G(i)-restricted signaling that inhibits water absorption in the collecting duct. J Biol Chem 280:35028-35037.
Heaslip & Sickels, 1989. Evidence that prostaglandins can contract the rat aorta via a novel protein kinase C-dependent mechanism. J. Pharmacol. Exp. Ther. 250, 44-51.
Hoffman et al. (1982) Effects of prostaglandins on the cerebral circulation in the coat. Prostaglandins 23:897-905.
ladecola et al., 2001. Reduced susceptibility to ischemic brain injury and N-methyl D-aspartate-mediated neurotoxicity in cyclooxygenase-2-deficient mice. Proc. Natl. Acad. Sci. U.S.A. 98, 1294-1299.
Jadhav et al. (2004) EP1- and EP3-receptors mediate prostaglandin E2-induced constriction of porcine large cerebral arteries. J Cereb Blood Flow Metab 24:1305-1316.
Jovanovic et al. (2006) An unexpected negative inotropic effect of prostaglandin F2alpha in the rat heart. Prostaglandins Other Lipid Mediat 80:110-119.
Kawano et al. (2006) Prostaglandin E2 EP1 receptors: downstream effectors of COX-2 neurotoxicity. Nat Med 12:225-229.
Kelley-Hickie & Kinsella (2004) EP1- and FP-mediated cross-desensitization of the alpha (alpha) and beta (beta) isoforms of the human thromboxane A2 receptor Br J Pharmacol 142:203-221.
Kiriyama et al., 1997. Ligand binding specificities of the eight types and subtypes of the mouse prostanoid receptors expressed in Chinese hamster ovary cells. Br. J. Pharmacol. 122, 217-224.
Kitanaka et al., 1994. Cloning and expression of a cDNA for rat prostaglandin F2 alpha receptor. Prostaglandins 48, 31-41.
Knock et al. (2005) Modulation of PGF2alpha- and hypoxia-induced contraction of rat intrapulmonary artery by p38 MAPK inhibition: a nitric oxidedependent mechanism. Am J Physiol Lung Cell Mol Physiol 28911039-1048.
Lerea et al., 1997. Prostaglandin F 2alpha is required for NMDA receptor-mediated induction of c-fos mRNA in dentate gyms neurons. J Neuroscience 17, 117-124.
Li et al., 1995. Up-regulation of brain PGE2 and PGF2 alpha receptors and receptor—coupled second messengers by cyclooxygenase inhibition in newborn pigs. J. Pharmacol. Exp. Ther. 272, 15-19.
Li et al., 2007. Neuronal Cyclooxygenase-2 Activity and Prostaglandins PGE2, PGD2, and PGF2alpha Exacerbate Hypoxic Neuronal Injury in Neuron-enriched Primary Culture. Neurochem. Res.
Li et al., 2004. Chronic behavioral testing after focal ischemia in the mouse: functional recovery and the effects of gender. Exp. Neurol. 187, 94-104.
Liu et al., 2005. Neuroprotection by the PGE2 EP2 receptor in permanent focal cerebral ischemia. Ann. Neurol. 57, 758761.
Lynch & Guttmann, 2002. Excitotoxicity: perspectives based on N-methyl-D-aspartate receptor subtypes. J. Pharmacol. Exp. Ther. 300, 717-723.
Mahagne (2007) [Acute management and secondary prevention of ischemic stroke]. Presse Med 36:128-133.
Manabe et al. (2004) Prostanoids, not reactive oxygen species, mediate COX-2-dependent neurotoxicity. Ann Neurol 55:668-675.
Mandal et al. (2005) Uteroglobin inhibits prostaglandin F2alpha receptor-mediated expression of genes critical for the production of pro-inflammatory lipid mediators. J Biol Chem 280:32897-32904.
Manabe, et al., 2004. Prostanoids, not reactive oxygen species, mediate COX-2-dependent neurotoxicity. Ann. Neurol. 55, 668-675.
Minegishi et al. (2002) Reactive oxygen species mediate leukocyte-endothelium interactions in prostaglandin F2alpha-induced Iuteolysis in rats. Am J Physiol Endocrinol Metab 283-E1308-1315.
Muller et al., 2000. Expression of PGF(2alpha) receptor mRNA in normal, hyperplastic and neoplastic skin. Carcinogenesis 21, 1063-1066.
Muralikrishna et al. (2006) Phospholipase A(2), reactive oxygen species, and lipid peroxidation in cerebral ischemia. Free Radic Biol Med 40:376387.
Nakahata et al., 2006. Vasodilation Mediated by Inward Rectifier K⁺ Channels in Cerebral Microvessels of Hypertensive and Normotensive Rats. Anesth. Analg. 102, 571-576.
Narumiya et al., 1999. Prostanoid receptors: structures, properties, and functions. Physiol. Rev. 79, 1193-1226.
Nonogaki et al., 1991. Dissociation of hyperthermic and hyperglycemic effects of central prostaglandin F2 alpha. Prostaglandins 41, 451-462.
Ogawa et al. (2007) Ischemia-induced neuronal cell death and stress response. Antioxid Redox Signal 9:573-587.
Ohtaki et al. (2006) Controlled normothermia during ischemia is important for the induction of neuronal cell death after global ischemia in mouse. Acta Neurochir Suppl 96:249-253.
Olson & Ammann (2007) Role of the prostaglandins in labour and prostaglandin receptor inhibitors in the prevention of preterm labour. Front Biosci 12:13291343.
Ota et al., 2005. The Effects of Prostaglandin Analogues on IOP in Prostanoid FP-Receptor-Deficient Mice. Invest. Opthalmol. Vis. Sci. 46, 4159-4163.
Perry et al., 2003. Latanoprost: An Update of its Use in Glaucoma and Ocular Hypertension. Drugs & Aging 20, 597-630.
Roy & Ray (2004) Effect of body temperature on mortality of acute stroke. J Assoc Physicians India 52:959-961.
Sharif et al. (2003) Ocular hypotensive FP prostaglandin (PG) analogs: PG receptor subtype binding affinities and selectivities, and agonist potencies at FP and other PG receptors in cultured cells. J Ocul Pharmacol Ther 19:501-515.
Shinohara (2006) Regional differences in incidence and management of stroke—is there any difference between Western and Japanese guidelines on antiplatelet therapy? Cerebrovasc Dis 21 Suppl 1:17-24.
Sjoquist et al., 2002. Ocular and systemic pharmacokinetics of latanoprost in humans. Sury Opthalmol 47 Suppl 1, S6-12.
Stjernschantz & Alm, 1996. Latanoprost as a new horizon in the medical management of glaucoma. Curr Opin Opthalmol 7, 11-17.
Spence, 2007. Combining multiple approaches for the secondary prevention of vascular events after stroke: a quantitative modeling study. Stroke 38, 1881-1885.
Sugimoto et al. (1997) Failure of parturition in mice lacking the prostaglandin F receptor. Science 277:681-683.
Suzuki-Yamamoto et al., 1999. cDNA cloning, expression and characterization of human prostaglandin F synthase. FEBS Lett. 462, 335-340.
Takayama et al. (2005) Thromboxane A2 and prostaglandin F2alpha mediate inflammatory tachycardia. Nat Med 11:562-566.
Toh et al., 1995. Molecular evolution of receptors for eicosanoids. FEBS Lett 361, 17-21.
Ueno et al., 1982. Role of prostaglandin D2 in the hypothermia of rats caused by bacterial Iipopolysaccharide. Proc. Natl. Acad. Sci. U.S.A. 79, 6093-6097.
Xu et al. (2005) Effect of glutamate on inflammatory responses of intestine and brain after focal cerebral ischemia. World J Gastroenterol 11:733736.
Xu et al., 2007. FP Prostanoid Receptor Mediated Induction of the Expression of Early Growth Response Factor-1 by Activation of a Ras/Raf/MAPK Signaling Cascade. Mol Pharmacol, DO I: 10.1 124/mol. 1 107.038778.

1. A method for identifying an agent that may be useful in the treatment or prevention of ischemic brain injury, comprising:
contacting an FP receptor polypeptide and PGF_{2α} with a test compound under conditions where the PGF_{2α} binds to the FP receptor polypeptide;
determining whether the test compound disrupts binding of the PGF_{2α} to the FP receptor polypeptide; and
determining whether the test compound prevents or decreases a symptom of ischemic brain injury in an animal model.

2. A method for identifying an agent that may be useful in the treatment or prevention of ischemic brain injury, comprising:
contacting an FP receptor polypeptide with a test compound;
determining whether the test compound binds to the FP receptor polypeptide; and
determining whether the test compound prevents or decreases a symptom of ischemic brain injury in an animal model.

3. A method for identifying an agent that may be useful in the treatment or prevention of ischemic brain injury, comprising:
contacting PGF_{2α} with a test compound;
determining whether the test compound binds to the GF_{2α}; and
determining whether the test compound prevents or decreases a symptom of ischemic brain injury in an animal model.

4. A method for identifying an agent that may be useful in the treatment or prevention of ischemic brain injury, comprising:
contacting an FP receptor polypeptide and PGF_{2α} with a test compound under conditions where the PGF_{2α} binds to the FP receptor polypeptide and induces PGF_{2α}-mediated signaling of the FP receptor polypeptide;
determining whether the test compound disrupts the PGF_{2α}-mediated signaling; and
determining whether the test compound prevents or decreases a symptom of ischemic brain injury in an animal model.
